(19) United States
(12) Patent Application Publication
(10) Pub. No.: US 2024/0368725 A1
(43) Pub. Date: Nov. 7, 2024

(54) COMPOSITIONS AND METHODS FOR BIOLOGICAL PRODUCTION AND HARVEST OF PRECIOUS METALS, PLATINUM GROUP ELEMENTS, AND RARE EARTH ELEMENTS

(71) Applicant: ECOBIOME HOLDINGS, LLC, THE WOODLANDS, TX (US)

(72) Inventor: MARC RODRIGUEZ, THE WOODLANDS, TX (US)

(73) Assignee: ECOBIOME HOLDINGS, LLC, THE WOODLANDS, TX (US)

(21) Appl. No.: 18/254,461

(22) PCT Filed: Dec. 3, 2021
(86) PCT No.: PCT/US2021/061860

§ 371 Date: May 25, 2023

Related U.S. Application Data
(60) Provisional application No. 63/120,997, filed on Dec. 3, 2020.
(60) Provisional application No. 63/196,509, filed on Jun. 3, 2021.

(51) Int. Cl. C22B 3/18; C22B 11/00; C22B 59/00

(52) U.S. Cl. CPC C22B 3/18; C22B 11/04; C22B 59/00

(57) ABSTRACT

The present disclosure provides an isolated Thiomonas isabelensis (ECOAU001) strain deposited at the Agricultural Research Service Culture Collection under the Accession number NRRL No. B-67995 according to the Budapest Treaty. The disclosed newly discovered bacterial strain is useful in precious metal extraction, production, and amplification. In particular, the present disclosure is directed to a novel microorganism useful to extract, produce and/or amplify precious metals and/or rare earth metals from an environmental substrate that it is cultured in.

### CROSS REFERENCE TO RELATED APPLICATIONS

This application is entitled to and claims the benefit, under 35 U.S.C. § 119 (e) of earlier filed U.S. Provisional Application Nos. 63/120,997, filed Dec. 3, 2020, and 63/196,509, filed Jun. 3, 2021, and claims priority under 35 U.S.C. § 371 to earlier filed Patent Cooperation Treaty Application No. PCT/2021/061860, filed Dec. 3, 2021, which earlier filed provisional applications and PCT application are incorporated by reference herein in their entirety.

### FIELD OF THE TECHNOLOGY

The present disclosure is directed to the field of precious metal extraction, production, and amplification. In particular, the present disclosure encompasses an isolated microorganism modified relative to wild-type and useful to extract, produce and/or amplify precious metals and/or platinum group elements and/or rare earth metals from a substrate such as an environmental substrate.

### BACKGROUND

In recent years, the price of gold and other precious metals has continuously increased, lifted by robust demand and a shortage of precious metals supplies. Global demand for precious and rare earth metals continues to rise rapidly while the global supply is not growing quickly enough to match the increasing demand. Many nations, particularly those with developing economies, do not have reliable access to precious and rare earth metals. In addition, inefficiencies in the supply chain, from mining and refinement to distribution and usage, make the availability of precious and rare earth metals unpredictable for many.

Gold mine production totaled 3,531 tons in 2019, 1% lower than in 2018, according to the World Gold Council. This is the first annual decline in production since 2008. The growth in mine supply is expected to continue to slow or even decline slightly in the coming years, as existing reserves are exhausted, and new major discoveries become increasingly rare.

The increase of demand for clean energy sources and growing popularity of electric vehicles and energy storage systems in the world may lead to an increased shortage of metals in the global market followed by a sharp increase in prices in years to come. According to recent estimates of the International Energy Agency (IEA), the demand for lithium will grow more than 40 times by 2040, while for cobalt and nickel by 20 times within the next two decades. The same situation is expected to be observed in the case of other precious metals.

Therefore, a need in the art exists for compositions and improved methods to extract, produce and/or amplify precious metals and/or rare earth metals.

### SUMMARY

Among the various aspects of the present disclosure provide compositions comprising an isolated modified bacterial strain Thiomonas isabelensis. In some embodiments, the bacterial strain is ECOAU001, which has been designated Accession number NRRL No. B-67995, deposited in accordance with the Budapest Treaty at the Agricultural Research Service Culture Collection (USDA, ARS, 1815 North University Street, Peoria, IL, 61064) on Nov. 13, 2020.

In one aspect, the disclosure provides a method of extracting precious metals and/or rare earth metals comprising contacting a solid substrate with a composition comprising an isolated modified bacterial strain Thiomonas isabelensis. In some embodiments, the bacterial strain is ECOAU001. In some embodiments, the composition includes one or more microbes used in industrial mining and as described herein.

In another aspect, the disclosure provides a method of amplifying precious metals and/or rare earth metals comprising contacting a solid substrate with a composition comprising an isolated modified bacterial strain Thiomonas isabelensis. In some embodiments, the bacterial strain is ECOAU001. In some embodiments, the composition includes one or more microbes used in industrial mining.

In one aspect, the disclosure provides a method of recovering precious metals and/or rare earth metals contacting a solid substrate with a composition comprising an isolated modified bacterial strain Thiomonas isabelensis. In some embodiments, the bacterial strain is ECOAU001. In some embodiments, the composition includes one or more microbes used in industrial mining.

In each of the preceding embodiment, the solid substrate can be a geological substrate, such as one or more of sandstone, limestone, shale, coal, chalk deposit formations, refractory rock ore or a solid substrate obtained from one or more of a terrestrial, aquatic or marine source. In each of the preceding embodiment, the solid substrate can be one or more of soil, biofilm, sediment, native metal rock and sludge residue.

In still another aspect, the disclosure provides a method of extracting precious metals and/or rare earth metals from a liquid substrate comprising contacting the liquid substrate with a composition comprising an isolated modified bacterial strain Thiomonas isabelensis. In some embodiments, the bacterial strain is ECOAU001. In some embodiments, the composition includes one or more microbes used in industrial mining.

In another aspect, the disclosure provides a method of amplifying precious metals and/or rare earth metals from a liquid substrate comprising contacting the liquid substrate with a composition comprising an isolated modified bacterial strain Thiomonas isabelensis. In some embodiments, the bacterial strain is ECOAU001. In some embodiments, the composition includes one or more microbes used in industrial mining.

In an aspect, the disclosure provides a method of condensing precious metals and/or rare earth metals from a liquid substrate comprising contacting the liquid substrate with a composition comprising an isolated modified bacterial strain Thiomonas isabelensis. In some embodiments, the bacterial strain is ECOAU001. In some embodiments, the composition includes one or more microbes used in industrial mining.

In some embodiments, the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm.

In some embodiments, the liquid substrate is obtained from one or more of a waste waters, sludge waters, saltwater, freshwater, irrigation systems, ponds, lakes, rivers, and estuaries source. In each of the preceding embodiments, the substrate is disinfected and/or sterilized prior to inoculation with the bacterial strain.

In some embodiments, the methods further comprising adding a fertilizer, nutrient and/or by product composition one or more times to the substrate after inoculation with the bacterial strain.

In some embodiments, the methods of the disclosure include allowing sufficient time for the bacterial strain to colonize and exponentially grow on or in the substrate. In some embodiments, the methods of the disclosure comprise using an anodic and cathodic LED having a wavelength generator set at a range of 2.0-22.0 KHz in the liquid substrate.

In some embodiments, the methods of the disclosure comprise harvesting the precious metals and/or rare earth metals by separating the precious metals and/or rare earth metals from the substrate.

In another aspect, the disclosure provides a composition comprising an isolated modified bacterial strain Thiomonas isabelensis, about 0.01%/Wt gold emulsion, about 0.01%/Wt gold nanoparticles, about 0.01%/Wt. adenosine triphosphate, about 1%/Wt. seaweed extract, about 0.5%/Wt. humic acid, about 0.5%/Wt. mixture of nitrogen, phosphate, potassium and micronutrient mixture, about 0.1% light and mid chain hydrocarbon mixture, about 0.25%/Wt. green solvent, about 0.75%/Wt. sugar, about 1.0%/Wt. substrate selected from a gold, precious metal group, platinum metal group, rare earth metal group and combinations thereof, and a carrier selected from water or agrose.

In some embodiments, the water carrier is selected from deionized water, distilled water, filtered water, well water, tap water, fresh water, sea water, brackish water, mineralized water, carbonated water, saline water, ionically charged water, ionized water, hydrogen water and combinations thereof.

In some embodiments, the composition further comprising one or more components selected from biosolvents ethyl lactate, ATP, ADP, pyrophosphate, soy based solvents, chemical solvents, green solvents, range of organic acids, lactic acid, malic acid, ascorbic acid, alkanes, alkenes, alkynes, saturates, aromatics, resinoids, asphaltenes, light, mid chain and heavy chain hydrocarbons, sodium nitrate, sodium nitrite, ethanol, sulfur, sulfate, sulfite, nitrogen, chemical surfactants (ionic, anionic, cationic, zwitterionic surfactants), polymers (low, mid, heavy chains), biosurfactants, glycolipids, rhamnolipids (J1 and J2), glycerin, propylene glycol, carbon sugars, dextrose, galactose, sucrose, fructose, complex carbohydrates, starch, cellulose, lignin, keratin, proteins and amino acids, manures, composts, green waste, sludge material, humic and fulvic acids, coal ash and coal derived waste, alumina cytokinins and seaweed extracts.

The details of one or more embodiments of the disclosure are set forth in the description below. Other features or advantages of the present invention will be apparent from the following drawings and detailed description of several examples, and from the appended claims.

### BRIEF DESCRIPTION OF THE FIGURES

Those of skill in the art will understand that the drawings, described below, are for illustrative purposes only. The drawings are not intended to limit the scope of the present teachings in any way.

FIG. 1A-1C show ECOAU1 modified activity relative to wild-type isolate in diverse environmental conditions. FIG. 1A shows the microbial respiration assay. FIG. 1B shows microbial urease enzyme assay. This assay measures the activity of the urease enzyme. Urease is a protein enzyme that catalyzes the hydrolysis of urea into NH₃ and CO₂ Reaction: NH₂—CO—NH₂+H₂O→NH₃+NH₂—COOH→NH₂+CO₂. ECOAU1 can effectively degrade urea-based fertilizers for enhanced nitrogen uptake. FIG. 1C shows microbial phosphorus solubilization assay. This assay measures the ability of a microorganism to cleave phosphorus from a compound and then metabolize that compound. All microbial strains are effectively able to degrade and release phosphorus from the soil for enhanced phosphorus plant root uptake. FIG. 1D shows microbial hydrogen sulfide assay. This assay measures the ability of a microorganism to metabolize sulfur compounds and produce H₂S and gas. All microorganisms do not produce the hydrogen sulfide in a low oxygen environment, thus are able to efficiently metabolize nutrients with substitute electron acceptors besides oxygen for efficient plant growth promotion.

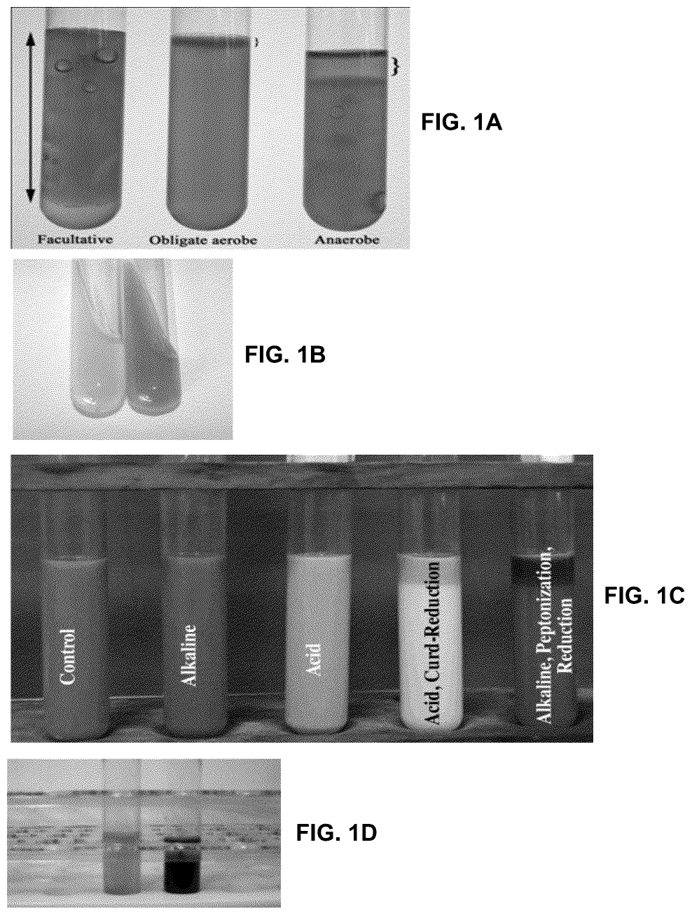

FIG. 2 show bioreactor and treatment spectrum for pressed soil with photon spectrum with Ti, AU, and Zn peaks identified.

FIG. 3 shows fire assay data of ECOAU001 treated versus control. Treated samples measured on average 7.44 ppm Au; a 76% increase from control's 4.19 ppm. The 95% confidence interval of the average sample mean is between 5.512647 ppm at the lower end and 9.367353 ppm at the high end. Control samples average 4.19 ppm.

FIG. 4 shows the percent cyanide recoverable gold in control versus ECOAU001 treated double refractory ore samples.

FIG. 5 shows the preg-rob factor percent in control versus ECOAU001 treated double refractory ore samples.

FIG. 6 shows the sulfide percent in control versus ECOAU001 treated double refractory ore samples.

FIG. 7 shows the percent cyanide recoverable gold in control versus ECOAU001 treated double refractory ore samples.

### DETAILED DESCRIPTION

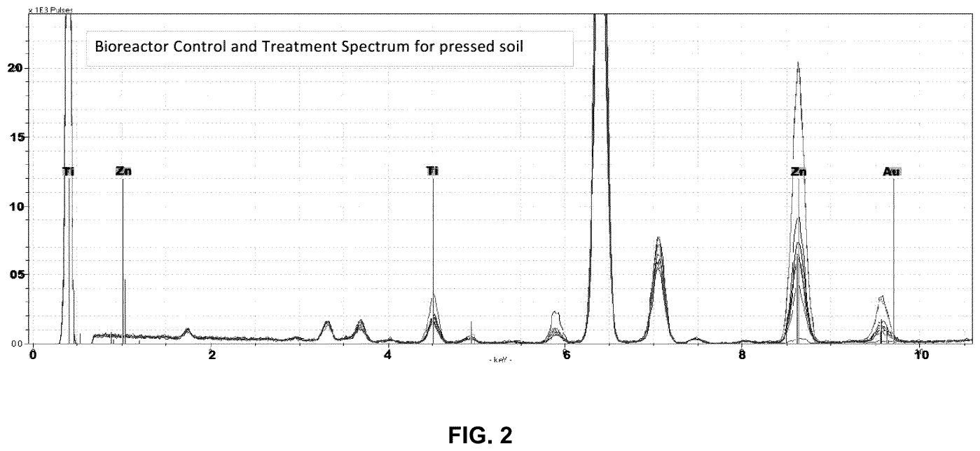

The present disclosure is based, at least in part, on the discovery of a bacterium isolated from the EcoBiome Innovation and Discovery apparatus. In brief, a rare earth mining sample originating from near Austin, TX was obtained and prepared for culturing in the Ecobiome apparatus. The Ecobiome apparatus and methods of use thereof are described in U.S. patent application Ser. No. 16/258,112 and is herein incorporated by reference in its entirety. The rare earth mining sample was crushed and loaded into the Ecobiome for microbial gradation and speciation. The prepped rare earth mining sample was allowed to equilibrate, and microbial growth promoted. After some time, a population of microorganisms were isolated, including a newly discovered wild-type bacteria. After isolation, the wild-type bacteria was subjected to a number of extreme conditions culture conditions, such as high and low pH, high and low temperature, and high and low salinity conditions which minimally forced enzymatic change (e.g., nitrate reductase, ligninase, cellulase, chitinase, and/or urease) thereby producing a modified bacterial strain relative to wild-type. Thus, as used herein, the term “modified” refers to a bacterial strain that has been forced to change, minimally, with respect to enzymatic gain of function. See, for example, FIG. 2 and the below examples.

Modification of the originally isolated wild-type bacteria resulted in the isolation and characterization of ECOA00U1 (Thiomonas isabelensis). Through various characterization methods it was found that the ECOA00U1 facilitated precious metal, rare earth and gold production from a variety of environmental solid and liquid substrata. The isolated ECOA00U1 demonstrated gold and precious metals production de novo, using eco-friendly and sustainable biochemical processes and methods.

Altogether, the present disclosure provides multiple lines of evidence showing the presently disclosed bacterium and methods of using the same to extract, produce and/or amplify precious metals and/or rare earth metals from a variety of environmental substrates. Other aspects and iterations of the invention are described more thoroughly below.

The bacterial strain disclosed in this description has been deposited under conditions that assure that access to the cultures will be available during the pendency of this application. The bacterial strain disclosed in this description has been deposited in the Agricultural Research Service Culture Collection (USDA, ARS, 1815 North University Street, Peoria, Ill., 61064). The bacterial strain deposited was designated as Thiomonas isabelensis. The deposit was received by the NRRL on Nov. 13, 2020 and was given an accession number by the International Depository Authority of B-67995. The deposit has been made to and received by the International Depository Authority under the provisions of the Budapest Treaty, and all restrictions upon public access to the deposit will be irrevocably removed upon the grant of a patent on this application. The deposits will be available as required by foreign patent laws in countries wherein counterparts of the subject application, or its progeny, are filed. However, it should be understood that the availability of the deposits does not constitute a license to practice the subject invention.

Further, the subject culture deposits will be stored and made available to the public in accord with the provisions of the Budapest Treaty for the Deposit of Microorganisms, i.e., they will be stored with all the care necessary to keep them viable and uncontaminated for a period of at least five years after the most recent request for the furnishing of a sample of a deposit, and in any case, for a period of at least thirty (30) years after the date of deposit or for the enforceable life of any patent which may issue disclosing the cultures. The depositor acknowledges the duty to replace the deposit(s) should the depository be unable to furnish a sample when requested due to the condition of the deposits.

Accordingly, one aspect of the present disclosure encompasses an isolated bacteria strain Thiomonas isabelensis (ECOAU001) which is modified relative to wild-type. Another aspect of the disclosure provides a mutant or derivative of ECOAU001 having the ability to extract, produce and/or amplify precious metals and/or rare earth metals as described herein. The term “mutant or derivative” thereof includes naturally occurring and artificially induced mutants which retain their ability to extract, produce and/or amplify precious metals and/or rare earth metals. Production of such mutants or derivatives will be well known by those skilled in the art including transgenic expression of heterologous nucleic acid sequences and/or genomic modifications.

In another aspect, the present disclosure provides compositions comprising ECOAU001. The concentration of ECOAU001 will vary depending on the type of composition. Suitable ECOAU001 concentrations include but are not limited to at least about 0.5×10¹⁰ CFU/Gm, least about 1.0×10¹⁰ CFU/Gm, least about 1.5×10¹⁰ CFU/Gm, least about 2.0×10¹⁰ CFU/Gm, least about 2.5×10¹⁰ CFU/Gm, least about 3.0×10¹⁰ CFU/Gm, least about 3.5×10¹⁰ CFU/Gm, least about 4.0×10¹⁰ CFU/Gm, least about 4.5×10¹⁰ CFU/Gm, least about 5.0×10¹⁰ CFU/Gm or greater.

A composition comprising ECOAU001 according to the present disclosure may comprise one or more additional components, including but not limited to, biosolvents ethyl lactate, ATP, ADP, pyrophosphate, soy based solvents, chemical solvents, green solvents, range of organic acids, lactic acid, malic acid, ascorbic acid, alkanes, alkenes, alkynes, saturates, aromatics, resinoids, asphaltenes, light, mid chain and heavy chain hydrocarbons, sodium nitrate, sodium nitrite, ethanol, sulfur, sulfate, sulfite, nitrogen, chemical surfactants (ionic, anionic, cationic, zwitterionic surfactants), polymers (low, mid, heavy chains), biosurfactants, glycolipids, rhamnolipids (J1 and J2), glycerin, propylene glycol, carbon sugars, dextrose, galactose, sucrose, fructose, complex carbohydrates, starch, cellulose, lignin, keratin, proteins and amino acids, fertilizer NPK (e.g., organic and inorganic fertilizers), manures, composts, green waste, sludge material, humic and fulvic acids, coal ash and coal derived waste, alumina cytokinins and seaweed extracts.

A composition comprising ECOAU001 according to the present disclosure may comprise a water source for microbial culturing or final product carrier. Non-limiting examples include deionized water, distilled water, filtered water, well water, tap water, fresh water, sea water, brackish water, mineralized water, carbonated water, saline water, ionically charged water, ionized water, and hydrogen water. Thus, according to the present disclosure compositions comprising microorganisms of the disclosure for use within the methods of the disclosure may comprise a water source for microbial culturing or final product carrier. Non-limiting examples include deionized water, distilled water, filtered water, well water, tap water, fresh water, sea water, brackish water, mineralized water, carbonated water, saline water, ionically charged water, ionized water, and hydrogen water. The aqueous solution may contain sufficient nutrients to support microbial growth. The useful nutrients are both inorganic and organic compounds commonly used to grow and nourish microbes. Inorganic nutrients include nitric acid, ammonium nitrate, ammonium chloride, ammonium sulfate, sodium nitrate, sulfur, sodium sulfide, sodium chloride, sodium bicarbonate, sodium phosphate, potassium phosphate, sulfuric acid, nitric acid, cyanide, uranium, mercury, lead, lithium, sodium metabisulfite, ammonium nitrate, fertilizers, gluconic acid, phosphogypsum, ferric chloride, calcium chloride, and ammonium phosphate. Organic nutrients include microbial biomass, glucose, dextrose, sodium acetate, amino acids, and purines. Vitamins that can be included in the nutrient solution include pyridoxine, pyridoxamine-HCl, riboflavin, thiamine, niacin, pantothenic acid, p-aminobenzoic acid, folic acid, and biotin. Small amounts of trace elements such as iron, copper, molybdenum and zinc can also be provided in the nutrient solution. Useful nutrients can also be mineral ores used for recovery of metals.

A composition comprising ECOAU001 according to the present disclosure may be formulated as a soil mixture, liquid, sludge or slurry substrate.

In some embodiments, a composition comprising ECOAU001 according to the present disclosure may comprise sulfuric acid, nitric acid, cyanide, uranium, mercury, lead, lithium, sodium metabisulfite, ammonium nitrate, fertilizers, gluconic acid, or phosphogypsum.

In one aspect, an ECOAU001 composition of the present disclosure may comprise Thiomonas isabelensis (ECOAU001 or ECOAU1) at a concentration of about 2.5×10¹⁰ CFU/Gm (5.0%/Wt.), gold emulsions at a concentration of about 0.01%/Wt., gold nanoparticles at a concentration of about 0.01%/Wt., adenosine Triphosphate (ATP) at a concentration of about 0.01%/Wt., seaweed at a concentration of about 1%/Wt., humic acid at a concentration of about 0.5%/Wt., NPK and micronutrients at a concentration of about 0.5%/Wt., a mixture of light and mid chain hydrocarbons at a concentration of about 0.1%/Wt., green solvents at a concentration of about 0.25%/Wt., a carbon sugar source at a concentration of about 0.75%/Wt., a representative substrate derived from a Gold, Precious Metal group, Platinum Metal group and/or Rare Earth metal group at a concentration of about 1.0%/Wt.; and an inert carrier, for example, a liquid or agarose which makes up the remainder of the mass to 100% final Weight (%/Wt.).

Another aspect of the present disclosure is a method to extract, produce and/or amplify precious metals and/or rare earth metals comprising culturing suitable microbe or plurality of suitable microbes. Non-limiting examples of suitable microbes include acidophilic archaea such as Sulfolobus metallicus and Metallosphaera sedula; mesophilic bacteria of the genera Acidithiobacillus or Leptospirillum ferrooxidans; Pyrococcus furiosus; thermoacidophilic archaeon Sulfolobus (Metallosphaera sedula); and Pyrobaculum islandicum. These microorganisms are basically 10, belonging to Bacteria: Acidiphilium sp., Leptospirillum sp., Sulfobacillus sp., Acidithiobacillus ferrooxidans and Acidithiobacillus thiooxidans; and Archaea: Acidianus sp., Ferroplasma sp., Metallosphaera sp., Sulfolobus sp. and Thermoplasma sp.

In a preferred embodiment, the methods include the bacterium strain ECOAU001 or a mutant or derivative thereof. Without wishing to be bound by theory, the mode of action of producing precious metals and/or rare earth metals is due to the ECOAU001's innate ability to extract, produce and/or amplify precious metals from the environmental substrate that it is cultured in. Isolated ECOAU001 is found to express various proteins and biochemicals which are modulated by the base concentrations of precious metals and/or rare earth metals in its surrounding environment. Additionally, ECOAU001 generates a high concentration of biomass, or microbial colonies throughout the culturing process, that enable it to hold high rates of gold and precious metals within its intracellular matrix.

For purposes of this disclosure, the term “mineral” or “mineral ore” means a composition that comprises precious metal values. Thus, a mineral may be a mined mineral, ancient seabed deposit, ancient lakebed deposit, black sands, an ore concentrate, metal bearing sea water, and waste products, such as mining tails, industrial waste water, oil well brine, coal tars, oil shales, tar sands, and oil sands. Useful minerals contain trace amounts of precious metals. Trace amount means the detection limit or below detection limits of conventional assay procedures such as fire assay, AAS (atomic adsorption spectroscopy), ICP-MS (inductive coupled plasma-mass spectrometer), ICP-AES (atomic emission spectroscopy) and other spectroscopic instrumentation commonly used in analytical laboratories. Some spectroscopic methods can detect as little as 1 ppt (part per trillion) to 0.1 ppb (part per billion).

As used herein, the term “rare earth metals” or “RE” may refer to scandium (Sc), yttrium (Y), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and/or lutetium (Lu). As used herein, the term “precious earth metals” may refer to gold, silver, aluminium, rhenium, indium, platinum, gallium, germanium, ruthenium, rhodium, beryllium, palladium, osmium, iridium, tellurium, bismuth, platinum palladium, titanium, zinc, and zirconium.

In some embodiments, the methods of extracting, producing and/or amplifying precious metals and/or rare earth metals generally comprise farming the precious metals and/or rare earth metals including the steps of inoculating the bacterial strains disclosed herein on solid substrates or geological substrates. A geologic substrate is a surface (or volume) of sediment or rock where physical, chemical, and biological processes occur, such as the movement and deposition of sediment, the formation of bedforms, and the attachment, burrowing, feeding, reproduction, and sheltering of organisms. Non limiting examples of a geological substrate useful for the present disclosure include sandstone, limestone, shale, coal, chalk deposit formations, refractory rock ore (e.g., single, double and triple refractory rock ore). Additional solid substrates include, but are not limited to an environmental sample collected from any terrestrial, aquatic or marine source such as soil, biofilms, sediments (e.g. coral or other marine sediments, aquifer sediments and the like), native metal rocks and sludge residue. In some embodiments, the solid substrate is disinfected prior to inoculation of the bacterial strains disclosed herein. Disinfection techniques include but are not limited to steam, autoclave, oven, microwave, biocide and fungicide solutions. Additional substrates include but are not limited to animal manures, bauxite, base metals, calcium phosphate, calcium silicate, clays and silicates, aluminum oxide, diatomaceous earth, diammonium phosphate, erionite and zeolites, feldspar, flint, food wastes, granite, graphite, gypsum, humic and fulvic acids, marble, mica, molten rock and lava, monoammonium phosphate, potash, pumice, silica, slate, seaweed, talc and recycled electronics and commercial devices.

In some embodiments, ECOAU001 may be applied to any solid substrate in a rock, powder, granulated or broken form for improved precious metals, platinum metals and rare earth metal leaching and extraction. In some embodiments, solid substrates are used in traditional farming, specialty farming, potted and greenhouse farming, hydroponics and aeroponics techniques. In some embodiments, the solid substrate includes old or recycled electronic components or batteries.

In one embodiment, the biological process using microbes according to the disclosure is conducted in commercially available bioreactor consisting of a reactor having an agitation means. The agitation means can be mechanical stirring with a flat bladed impeller, percolation column, or air agitated pachuca reactor. The bioreactor can have air intake means, sterilization means, harvesting means, heating and/or cooling means, temperature controller means, pH controller means, filtration means and pressure controller means. All these features of bioreactors are known and commercially available in the biotechnology industry.

The biological process using microbes according to the disclosure can also be done by heap leaching techniques. In heap bio leaching techniques, a large body of mineral ore is treated with mutant microbes in nutrient solution in large contaminant ponds with no agitation and/or only occasional agitation. Generally, the contact time for heap type bio treatment is substantially longer than the agitated bioreactors and range from 10 days to 100 days.

As used herein, the term “inoculating” refers to the act of introducing a microorganism or a plurality of microorganisms (e.g. ECOAU001) into a substrate where it will be metabolically active and/or propagate. In preferred embodiments, the step of inoculating is performed using aseptic technique. In some embodiments, the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm. In a preferred embodiment, the bacterial strain is inoculated at a concentration from about 1.0×10⁶ CFU/gm to about 1.0×10¹² CFU/gm. The inoculating step can occur one or more times during the duration of extracting, producing and/or amplifying precious metals and/or rare earth metals from the solid substrate.

Inoculation of the solid substrate can occur by any means know to the skilled artisan which provides the microbe to the substrate in a sufficient amount. In some embodiments, after inoculation additional solid substrate is added to increase to surface area of the solid substrate which is in contact with the bacterial strain. In one aspect, additional solid substrate is added to create a 4-6 inch depth over the initial inoculation depth.

After inoculation the solid substrate is optionally irrigated and/or fertilized one or more times to stimulate colonization and exponential growth throughout of the bacterial strain throughout the solid substrate. Exemplary fertilizers include a low NPK plus micronutrient fertilizer, solutions comprising a complex or simple sugar, a seaweed or cytokine and a vitamin blend. In addition, specialty nutrients and by-products can be added to the inoculated solid substrate one or more times to establish new, increased and rigorous colonization by the bacterial strain.

In addition, the solid substrate may be covered to maintain a stable temperature or allow for an increase in the solid substrate temperature, for example using a poly covering for consistent temperature control and to control microbial contaminants from colonizing. In some embodiments, the inoculated solid substrate is maintained at a temperature between about 20° C. to about 60° C. including any range therein. In a preferred embodiment, the inoculated solid substrate is maintained at a temperature between about 29° C. to about 50° C.

After the bacterial strain has had sufficient time to colonize and biochemically process the solid substrate (non-limiting example 2-10 weeks) the solid substrate can be tested and processed for precious metals and/or rare earth metals production. The testing and/or processing steps include harvesting the solid substrate which has been colonized by the bacterial strain, generating a slurry by adding a solution to the solid substrate, and centrifugation at a minimum of 8,000 RPM to concentrate the precipitate which contains the de novo precious metals and/or rare earth metals. These steps may optionally include a bacterial lysis step to release any metals within the bacterial strains intracellular matrix.

In a still another aspect, the methods of extracting, producing and/or amplifying precious metals and/or rare earth metals generally comprise inoculating the bacterial strains disclosed herein in liquid substrates. Suitable liquid substrates include but are not limited to balanced salt and nutrient solutions, broths, environmental samples collected from any aquatic or marine source, waste waters, sludge waters, saltwater, freshwater, irrigation systems, ponds, lakes, rivers, and estuaries. In some embodiments, the liquid substrate is disinfected prior to inoculation of the bacterial strains disclosed herein. Disinfection techniques include but are not limited to steam, autoclave, oven, microwave, biocide and fungicide solutions. In a preferred embodiment, the disinfection step will reduce microbial colony and propagule concentrations to below or at about 5.0×10⁵ CFU/ml.

Inoculation of the liquid substrate can occur by any means known to the skilled artisan at concentration described above for the solid substrate. The inoculating step can occur one or more times during the duration of extracting, producing and/or amplifying precious metals and/or rare earth metals from the liquid substrate. After inoculation the liquid substrate is preferably agitated during the extraction, production and/or amplification of the precious metals and/or rare earth metals. In an exemplary embodiment, agitation of the liquid substrate occurs using an air pump for aerobic respiration.

After inoculation the liquid substrate is optionally specialty nutrients and by-products can be added to the inoculated solid substrate one or more times to establish new, increased and rigorous colonization by the bacterial strain, for example by adding solutions comprising a complex or simple sugar, a seaweed or cytokinin and a vitamin blend. In addition, for accelerated reactions establish an anodic and cathodic LED using a wavelength generator set at a range of 2.0-22.0 KHz.

After the bacterial strain has had sufficient time to colonization and biochemically process the liquid substrate (non-limiting example 12-72 hours) the liquid substrate can be tested and processed for precious metals and/or rare earth metals production. The testing and/or processing steps include collecting the liquid substrate which has been colonized by the bacterial strain, generating a slurry by adding a solution to the solid substrate, and centrifugation of the liquid solution through an in line and continuous centrifuge at a minimum of 8,000 RPM to concentrate the precipitation. These steps may optionally include a bacterial lysis step to release any metals within the bacterial strains intracellular matrix.

In each of the above embodiments, a bioreactor, fermenter, reaction vessel can be used in the disclosed methods. Moreover, the present disclosure contemplates the use of the disclosed microbes for bioleaching and heap leaching and therefore the use of leach pits are contemplated within the methods as well.

Platinum and its sister metals palladium and rhodium can be recovered using the methods according to the present disclosure by using pollution-cutting catalytic converters.

Bio treatment temperature ranges from 15 degrees centigrade to 50

degrees centigrade, preferably from 20 degrees to 30 degrees centigrade. pH can be acidic (pH 1 to 3) or basic (pH 9 to 12), although slightly acidic (pH 4) to slightly basic (pH 8) pH ranges are preferred. The most preferred pH ranges are the neutral range of from pH 6.5 to pH 7.5.

In accordance with the methods of the present disclosure, pressure is not critical and can be at atmospheric, below atmospheric, and/or above atmospheric. The biological transmutation process can be conducted in aerobic or anaerobic conditions. The biological transmutation process can be conducted in the presence of nitrogen, carbon dioxide, and oxygen in the atmosphere. Oxygen can be provided chemically, for example, with hydrogen peroxide, or as a gas from pressurized vessels.

Microbe concentration is not critical. At low microbe concentration, the contact duration is generally longer to allow the microbe to grow and multiply. However, microbe concentration should not exceed the maximum microbe concentration that the nutrient solution can sustain. Contact time can vary from a few hours to several weeks and depends in part on the type and mesh size of the mineral ore digested. Contact time ranges can be from 1 day to 30 days, more preferably from 1 day to 10 days.

The biological process using microbes according to the disclosure can be conducted in aerobic or anaerobic conditions. However, preferably conducted in the presence of oxygen, nitrogen and carbon dioxide in the atmosphere. Oxygen can also be provided chemically, for example, with hydrogen peroxide, or as a gas from pressurized vessels.

Nutrients can also be provided during the biological transmutation process to support growth of the mutant microbes. Nutrients can be inorganic, including nitric acid, sulfur, ammonium nitrate, ammonium chloride, ammonium sulfate, sodium nitrate, sodium chloride, sodium bicarbonate, sodium phosphate, potassium nitrate, potassium phosphate, ferric chloride, calcium chloride, and ammonium phosphate, and organic, including glucose, dextrose, sodium acetate, amino acids, and purines. Vitamins that can be included in the nutrient solution include pyridoxine, pyridoxamine-HCl, riboflavin, thiamine, niacin, pantothenic acid, p-aminobenzoic acid, folic acid, and biotin. Small amounts of traces elements such as iron, copper, molybdenum and zinc can also be provided in the nutrient solution.

In a still another aspect, the biological transmutation process using microbes according to the disclosure occurs in liquid substrates. Suitable liquid substrates include but are not limited to balanced salt and nutrient solutions, broths, environmental samples collected from any aquatic or marine source, waste waters, sludge waters, saltwater, freshwater, irrigation systems, ponds, lakes, rivers, and estuaries. In some embodiments, the liquid substrate is disinfected prior to inoculation of the bacterial strains disclosed herein.

Inoculation of the liquid substrate can occur by any means known to the skilled artisan at concentration described above for the solid substrate. The inoculating step can occur one or more times during the duration of extracting, producing and/or amplifying precious metals and/or rare earth metals from the liquid substrate. After inoculation the liquid substrate is preferably agitated during the extraction.

After inoculation the liquid substrate is optionally specialty nutrients and by-products can be added to the inoculated solid substrate one or more times to establish new, increased and rigorous colonization by the bacterial strain, for example by adding solutions comprising a complex or simple sugar, a seaweed or cytokinin and a vitamin blend. In addition, for accelerated reactions establish an anodic and cathodic LED using a wavelength generator set at a range of 2.0-22.0 KHz.

In each of the above embodiments, a bioreactor, fermenter, reaction vessel can be used in the disclosed methods. Moreover, the present disclosure contemplates the use of the disclosed microbes for bioleaching and heap leaching and therefore the use of leach pits are contemplated within the methods as well.

After the biological transmutation process, the recovery of metal produced from the starting material and microbial solution can be performed by conventional metallurgical methods such as smelting, leaching, electrolysis, resins and other methods known to those skilled in art of metallurgy. In another embodiment, the precious metals in the microbes or biomass of dead microbes can be recovered by methods described for recovery of precious metals from mineral ore.

Fire assaying and cupellation are described by C. W. Ammen, Recovery and Refining of Precious Metals, second edition 1993, Chapter 12, pp 302-329.

Also provided are kits. Such kits can include an agent or composition described herein and, in certain embodiments, instructions for use. Such kits can facilitate performance of the methods described herein. When supplied as a kit, the different components of the composition can be packaged in separate containers and admixed immediately before use. Components include but are not limited to ECOAUI compositions and formulations for use or stability, as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B. D. Hames & S. J. Higgins eds. (1985»; Transcription and Translation (B.D. Hames & S.J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

So that the present disclosure may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

In this disclosure, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Novel Microorganism Isolation and Classification

It has been estimated that only 2% of all microbial isolates can be cultured in a lab. Therefore, the microbes that can be grown in the laboratory represent only a small fraction of the total diversity that exists in nature. At all levels of microbial phylogeny, uncultured clades that do not grow on standard media are playing critical roles in cycling carbon, nitrogen, and other elements, synthesizing novel by-products, and impacting the surrounding organisms and environment. The ability to culture difficult to culture or previously uncultured microbial strains provides a wealth of information about their role in the environment, ecology, and nutrient cycling. But perhaps even more importantly, screening of novel isolates will reveal novel products that can have profound effects for the discovery of novel drugs, improve agricultural techniques and products, and in the production of rare and precious metals. To solve this problem the Applicant developed a novel apparatus (Ecobiome Discovery apparatus) allowing for the controlled growth, isolation and characterization of microorganisms including those that are difficult to culture or are uncultivable at the present time.

In an effort to isolate and characterize novel microbes, an experiment utilizing the Ecobiome Discovery apparatus was used. A rare earth mining sample originating from near Austin, TX was obtained and prepared for culturing in the Ecobiome apparatus. In short, the rare earth mining sample was crushed and loaded into the Ecobiome for microbial gradiation and speciation. The prepped rare earth mining sample was allowed to equilibrate and microbial growth promoted. After some time a population of microorganisms, including the newly discovered organism (ECOAU1) were isolated.

After isolation of the microbe population comprising ECOAU1, challenge tests were performed, which included tests specific for precious and rare metal remediation microbes. These test resulted in the isolation and characterization of ECOAU1 (Thiomonas isabelensis).

ECOAU1 is a gram-negative bacterium, non-spore former that is capable of metabolizing simple and complex polymers as well as metals through heterotrophic and chemoheterotrophic biochemical pathways. ECOAU1 is a facultative anaerobe and is tolerant of low and high temperatures, e.g., ranging between about 5° C. to about 46° C. In addition, ECOAU1 is tolerant of a pH range from about 2 to about 9. The microorganism reaches exponential growth with high agitation (>360 rpm) and oxygenation (DO>90%) within 6-8 hours.

### Example 2: Manufacturing Precious and Rare Earth Elements

Ten pounds of soil taken from a North Carolina riverbed was used as the processing soil for the trial. The ten pounds were weighed out and placed in a bucket container fashioned specifically for holding soil and dry material during processing in a bioreactor.

The bucket of soil was inserted into the bioreactor and the reactor was filled to 40 gallons of filtered water. Once the water level exceeded the height of the bucket the circulating water pump was turned on in addition to a continuous air pump into the bioreactor.

Prior to inoculation with the microorganism, a disinfection process was initiated using Calcium Hypochlorite followed by Ascorbic Acid neutralization. This allowed for the disinfection of any contaminant microorganisms that may have been present in water, soil or air contaminants to be removed.

After ascorbic acid neutralization of the bioreactor contents, the EcoBiome AU001 microorganism, labeled Thiomonas isabelensis (ECOAU001) was transferred and inoculated into the bioreactor at a rate of 1.0×10¹⁰ CFU/ml.

Nutrient contents were then added which consisted of a blend of NPK fertilizer, seaweed extract, zinc citrate, zinc sulfate, Vitamin blend, Dextrose and 3 liters of Nutrient Broth (autoclaved and sterilized).

The reaction was closed and sealed and allowed to react for 48 hours with an internal liquid temperature of approximately 40° C. using continuous aeration and recirculation.

Once the reaction was completed, all liquid contents were run through centrifugation, harvested, dried in an oven and then analyzed via an X-Ray Fluorescence Analyzer for precious metals and rare earth elements. % Gold production is reported in Table 1, below.

TABLE 1
EcoAU001 gold concentrations

| | |
|---|---|
| Bioreactor Trial | Gold (Au) % by Weight |
| | (X Ray Fluorescence Analyzer) |
| Untreated Control | 0 |
| | (Less than the Limit of Detection) |
| Sample 1 - Centrifugation 1x | 0.369 |
| Sample 2 - Centrifugation 2x | 0.984 |
| Sample 3 - Soil attached to | 3.676 |
| bucket screen | |

Average % by Weight extraction of Gold (Au) is in the range of 10 grams-15 grams per 2,000 pounds of rock and soil (0.000011%-0.000016% extraction efficiency rate). All (3) Trial samples significantly increased extraction efficiencies compared to the Untreated control soil.

As can be seen in Table 1, the untreated control (soil without ECOAU001 inoculation) lacked a gold concentration within the limit of detection by the analyzer. Sample 1 was completed and was run through the centrifuge at 8000 rpm 1 time. Sample 2 was the effluent after sample 1 was centrifuged and then run through the centrifuge a 2^{nd} time, to determine whether more material could be captured by the centrifuge. Sample 3 was located at the screens placed to hold the solid soil substrate. A layer of soil like material being captured on the screen throughout the process was identified. Therefore, this sample was taken for testing and called this the bucket screen sample (Sample 3).

As can be seen in Table 1, incubation of the riverbed material with ECOAU001 within the bioreactor resulted in the production and capture of gold at concentrations greater than could be detected in the untreated control. Centrifugation aided in capture of gold and the use of screens within the bioreactor to hold solid soil captured a soil-like material that showed the highest concentration of gold.

### Example 3: Microbial Gold And Precious Metals Farming (Includes Traditional Farming, Specialty Farming, Potted and Greenhouse Farming, Hydroponics and Aeroponics)—Soil, Native Metal Rocks and Geological Substrates

The starting soil is disinfected with a standard biocide and fungicide to reduce microbial colony and propagule concentrations to below or about 5.0×10⁵ CFU/Gm.

The soil was then inoculated using a soil drench, irrigation, or overhead irrigation treatment with ECOAU001 plus ECOAU001 nutrients and by-products at a minimum concentration CFU/Gm soil of about 1.0×10⁶ CFU/gm-1.0×10¹² CFU/Gm.

The soil was then tilled up to 4-6 inches to allow for greater surface area contact between the ECOAU001 microbes and the soil.

The soil was lightly irrigated and fertilized with a low NPK plus micronutrient fertilizer for stimulating ECOAU001 microbial colonization and exponential growth throughout the soil or substrate.

The treated soil was then covered with a tarp or cover to maintain a stable temperature or allow for an increase in the soil temperature to a range between about 29° C.-50° C.

The treated soil was lightly irrigated weekly or more frequently in some areas to improve colonization concentration and growth.

Post primary inoculation one or more additional inoculations of ECOAU001 plus specialty nutrients and by-products occurred every 2-4 weeks to re-establish new, increased and rigorous colonization by the microbes.

During the gold and precious metal production phase one or more tests were performed to confirm ECOAU001 mediated gold and precious metals production and extraction through standard precious metals testing and analysis.

Harvest the precious metals and gold occurred by removing the top 2-4 inches of soil, transferring to a contained area with a floor lining, adding water to the soil and bringing the mixture to a slurry. The slurry is then centrifuged through continuous centrifugation at a minimum of 8,000 RPM to concentrate the precipitate which includes contains the de novo gold and precious metals. This process was repeated thereby enriching for the de novo gold and precious metals.

### Example 4: Microbial and Precious Metals Extraction—Liquid Solutions and Substrates (Saltwater, Freshwater, Irrigation Systems, Ponds, Lakes, Rivers, Estuaries and Any Body of Water)

The starting water or starting liquid solution were disinfected with a standard biocide to reduce microbial colony and propagule concentrations to below or at about 5.0×10⁵ CFU/ml.

Freshwater, brackish and seawater samples were inoculated with ECOAU001 plus ECOAU001 nutrients and by-products at a minimum concentration CFU/ml liquid of about 1.0×10⁶ CFU/ml-1.0×10¹² CFU/ml. The inoculated water samples were then agitated using an air pump for aerobic respiration.

For accelerated reactions an anodic and cathodic LED was used using a wavelength generator set at a range of 2.0-22.0 KHz.

The inoculated water samples were then allowed to culture and produce precious metals for 24-48 hours.

To harvest the precious metals, the liquid solution was centrifuged through an in line and continuous centrifuge at a minimum of 8,000 RPM to concentrate the metal precipitation.

### Example 5: EcoBiome Analysis of Fire Assay Samples

The goal of this analysis was to determine whether EcoBiome's Rare Metals Division proprietary treatment produces a gold (Au) recovery rate statistically higher than the sample control content of 4.19 ppm. A sample of about 12 pounds from a gold mine was treated. Treated samples were sent to Hazen Research, Inc lab to measure Au content in ppm by the fire assay method. In this example the following 10 samples were analyzed.

TABLE 2
Sample Results

| | | | Gold in | Gold in |
|---|---|---|---|---|
| Sample ID | Treatment | Batch | ppm | g/ston |
| 21M01497-001 | Control | | 4.22 | 3.83 |
| 21M01497-002 | Centrifuge | | 3.39 | 3.08 |
| 21M01585-001 | Centrifuge | Mar. 8, 2021 | 10.30 | 9.34 |
| 21M01585-002 | Centrifuge | Mar. 8, 2021 | 7.68 | 6.97 |
| 21M01585-004 | Centrifuge | Mar. 22, 2021 b | 7.54 | 6.84 |
| 21M01585-006 | Control | | 4.18 | 3.79 |
| 21M01718-003 | Centrifuge | Apr. 5, 2021 (1) | 13.10 | 11.90 |
| 21M01718-004 | Centrifuge | Apr. 5, 2021 (2) | 10.70 | 9.71 |
| 21M01718-017 | Centrifuge | Apr. 12, 2021 (1) | 4.73 | 4.29 |
| 21M01718-018 | Centrifuge | Apr. 12, 2021 (2) | 7.03 | 6.38 |
| 21M01718-019 | Centrifuge | Apr. 12, 2021 (3) | 5.07 | 4.60 |
| 21M01718-020 | Centrifuge | Apr. 12, 2021 (4) | 4.87 | 4.42 |
| 21M01718-021 | Control | | 4.17 | 3.78 |

### Analysis

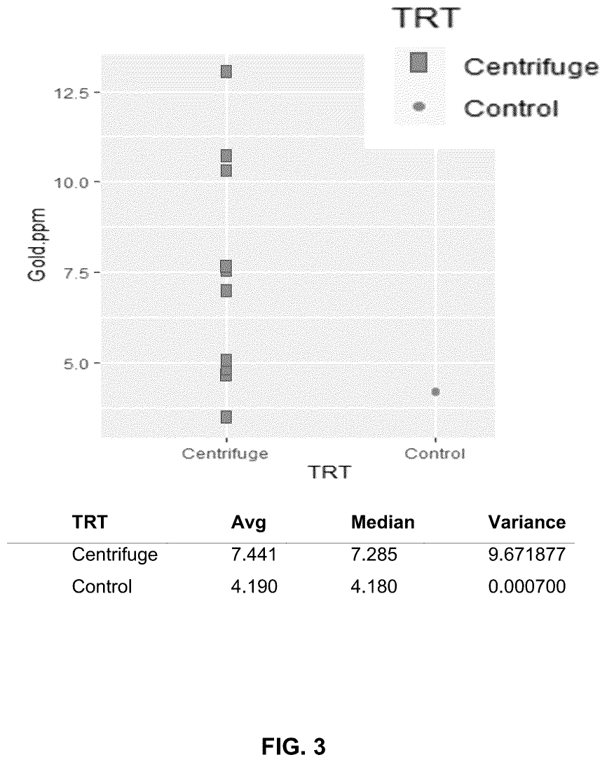

Treated samples measured on average 7.44 ppm Au; a 76% increase from control's 4.19 ppm. The 95% confidence interval of the average sample mean is between 5.512647 ppm at the lower end and 9.367353 ppm at the high end. Control samples average 4.19 ppm. Also, summarized are basic statistical measures, see e.g., FIG. 3 showing a graph depicting treatment versus control results and the table shows the average.

### Test Results

It was concluded that the average Au content in the treated sample is statistically significantly higher than control's 4.19 ppm. Moreover, on average, as expected, the treated samples to yield at least 5.63 ppm of Au, a 33% increase compare to the current industry standard of 4.22 ppm.

### Element and Metals Changes

Metals and elements were tracked as a response to changes in Gold concentrations. The following table lists average changes in metals content over 5 batch runs.

TABLE 3
Average Changes in Metals

| | | EcoBiome | |
|---|---|---|---|
| | Double | ‘Cultured | |
| | Refractory | GOLD’ | % Change |
| | Ore | (ppm; Post | (EcoBiome vs. |
| Elements | (ppm; As is) | Processing) | As Is Sample) |
| Copper | 89 | 261 | +293% |
| Iron | 18,844 | 14,138 | −25% |
| Magnesium | 12,547 | 0 | −100% |
| Sulfur | 24,043 | 15,060 | −37% |
| Aluminum | 72,742 | 36,747 | −50% |
| Arsenic | 3,516 | 804 | −77% |
| Barium | 484 | 252 | −48% |
| Selenium | 8 | 0 | −100% |
| Nickel | 23 | 0 | −100% |

5 Batch Averages

TABLE 4
Rare Earth Element Concentration Changes

| | % Increase Over |
|---|---|
| Element | Control (Mean) |
| Neodymium | +53% |
| Lanthanum | +42% |
| Yttrium | +41% |
| Cerium | +54% |
| Dysporium | +65% |
| Praseodymium | +63% |
| Scandium | +61% |
| Lutetium | +59% |

Method: ICP

### Example 6: Double Refractory Ore (Northern Nevada Gold Mine)—% RECOVERABLE CYANIDE GOLD

An untreated (As Is) sample of gold ore rock was processed through the EcoBiome Platform to demonstrate changes in the mobility of elemental gold. The standard processing of rock ore was initiated to determine if the addition of the Thiomonas isabelensis microorganism resulted in an increase in gold production and recovery.

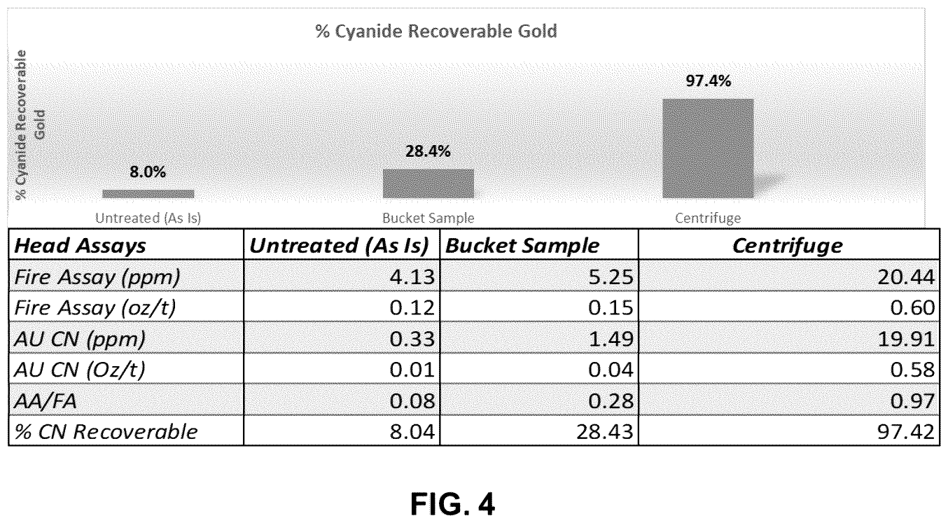

As shown in FIG. 4, the As Is untreated control demonstrated a gold recovery of 8.0%, whereas the Thiomonas isabelensis treated samples yielded higher and significant improvements in gold recovery and gold production as measured through the Fire Assay (ppm) and CN % recoverable percentages. The bucket sample yielded a 28.4% total recovery and the centrifuge sample yielded a 97.4% increase in recovery of gold from the rock ore sample.

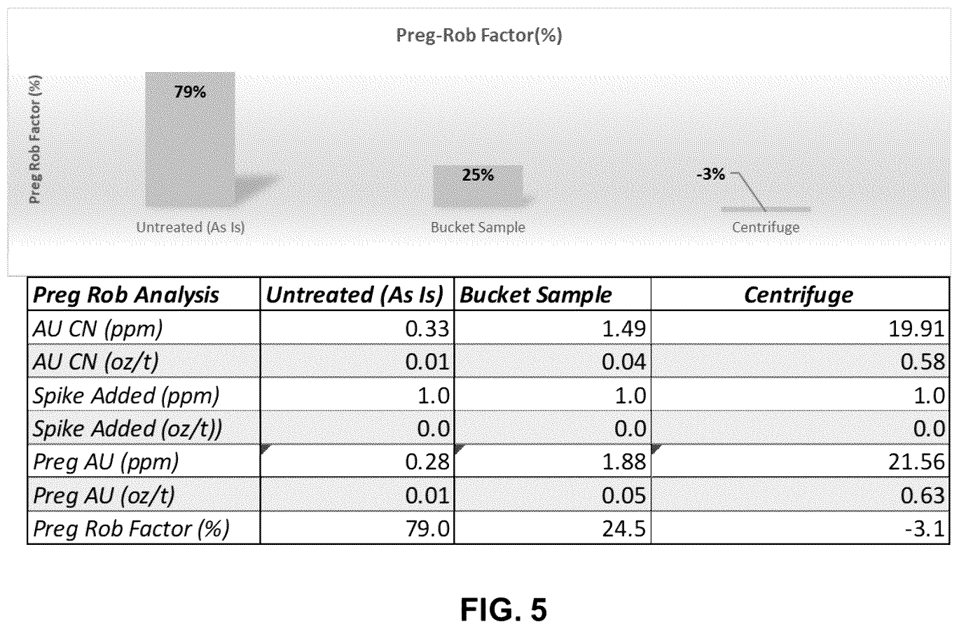

Gold ores associated with carbonaceous matter present several difficulties in their extraction due to the ability of this matter to adsorb the aurocyanide complex from gold pregnant solutions, resulting in low extractions by direct cyanidation. This phenomenon is known as preg robbing. To examine the preg rob factor %, standard cyanide processing of rock ore was used to compare against the biotechnology processing using the microorganism Thiomonas isabelensis. As shown in FIG. 5, the As Is untreated control demonstrated an organic carbon preg robbing factor of 7%, whereas the Thiomonas isabelensis treated samples yielded lower and significant reductions in preg robbing factors as measured through standard preg robbing tests. The bucket sample yielded a 25.0% total preg robbing factor and the centrifuge sample yielded a −3.0% reg robbing factor or reduction of organic carbon from the rock ore sample.

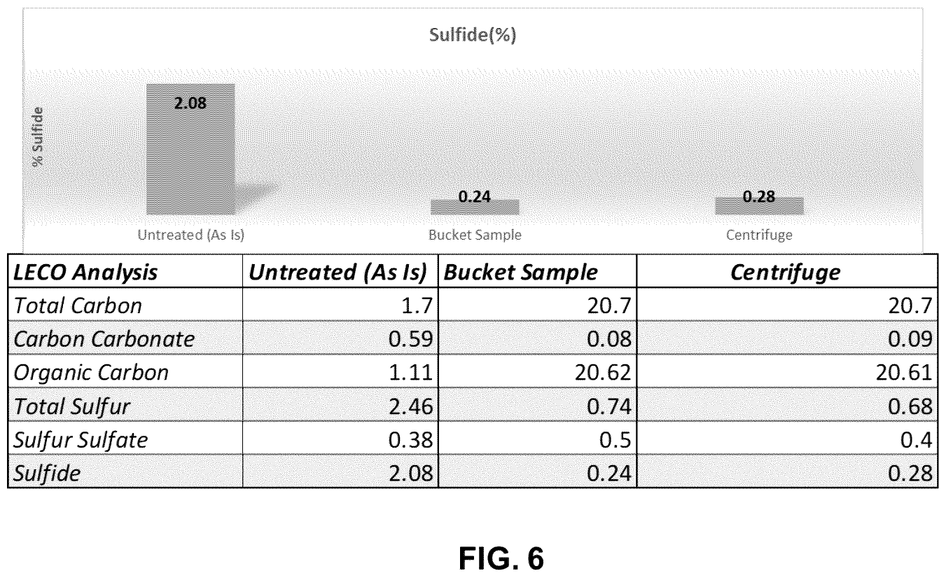

Moreover, examination was conducted to demonstrate and measure substantial changes in Sulfide % fund in the rock ore. As shown in FIG. 6, the As Is untreated control demonstrated a Sulfur Sulfide percentage of 7%, whereas the Thiomonas isabelensis treated samples yielded lower and significant reductions in Sulfur Sulfide percentages as measured through standard LECO and sulfide quantifying assays. The treated bucket sample yielded a 0.24% sulfide % and the treated centrifuge sample yielded a 0.28% sulfide % or reduction of organic carbon from the rock ore sample.

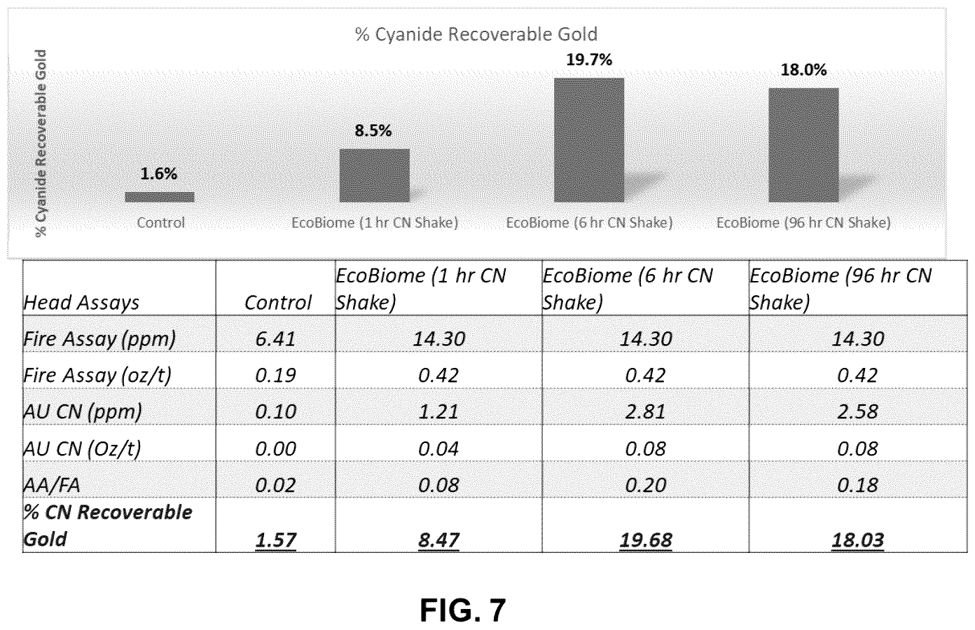

FIG. 7 shows the As Is untreated control demonstrated a gold recovery of 1.6%, whereas the EcoBiome Thiomonas isabelensis treated samples yielded higher and significant improvements in gold recovery and gold production as measured through the Fire Assay (ppm) and CN % recoverable percentages. The EcoBiome samples were measured after a 1 hr., 6 hr. and 96 hr. Cyanide Shake test increase in recovery of gold from the rock ore sample. The 1 hr. CN shake test yielded an 8.5% increase (14.30 ppm Fire Assay), the 6 hr. CN shake test yielded a 19.7% increase (14.30 ppm Fire Assay) and the 96 hr. CN shake test yielded a 18.0% increase (14.30 ppm Fire Assay) compared to the untreated control which yielded a 1.6% (6.41 ppm Fire Assay).

### Example 7: Forced Modification and Enzymatic Activity

Characterization of wild-type and modified ECOAU1. Microbial urease enzyme assay measures the activity of the Urease enzyme (Table: 5). Urease is a protein enzyme that catalyzes the hydrolysis of urea into NH₃ and CO₂. Microbial Respiration Assay measures the ability of a microorganism to utilize oxygen or an alternative final electron acceptor in its metabolic pathway. Microbial Phosphorus Solubilization Assay measures the ability of a microorganism to cleave phosphorus from a compound and then metabolize that compound. Microbial Hydrogen Sulfide Assay measures the ability of a microorganism to metabolize sulfur compounds and produce H₂S and gas. Microbial Chitinase Enzyme Assay measures the ability of a microorganism to produce an extracellular Chitinase enzyme that catabolizes chitin compounds in the soil. Microbial Cellulase Enzyme Activity measures the ability of a microorganism to produce an extracellular Cellulase enzyme that catabolizes cellulose and hemi-cellulose compounds in the soil. Microbial Ligninase Enzyme Activity measures the ability of a microorganism to produce an extracellular Ligninase enzyme that catabolizes lignin compounds in the soil.

Microbial chitinase enzyme assay measures the ability of a microorganism to produce an extracellular Chitinase enzyme that catabolizes chitin compounds in the soil. All microorganisms are able to degrade and decompose chitin, the second most abundant complex polymer in the soil and the primary component of fungal walls and nematode eggs, for improved soil mineralization. The microbial cellulase enzyme activity assay measures the ability of a microorganism to produce an extracellular Cellulase enzyme that catabolizes cellulose and hemi-cellulose compounds in the soil. ECOAU1 is able to effectively degrade and decompose cellulose, the most abundant complex polymer in the soil for increased mineralization and nutrient cycling. Cellulose is the building block of all stalk, stover and crop residue. The microbial ligninase enzyme activity measures the ability of a microorganism to produce an extracellular Ligninase enzyme that catabolizes lignin compounds in the soil.

TABLE 5
Microbial Urease Enzyme Assay

| Microorganism | Urease Enzyme Activity |
|---|---|
| wild type | − |
| Thiomonas isabelensis | ++ |
| ECOAU1 modified | |

Legend

| | |
|---|---|
| − | No Enzymatic Activity |
| + | Enzymatic Activity |
| ++ | Strong Enzymatic Activity |

TABLE 6
Microbial Respiration Assay

| Microorganism | Metabolic Oxygen Activity |
|---|---|
| wild type | Facultative Anaerobe |
| Thiomonas isabelensis | Facultative anaerobe |
| ECOAU1 modified | |

TABLE 7
Microbial Phosphorus Solubilization Assay

| Microorganism | Phosphorus Solubilization |
|---|---|
| wild type | + |
| Thiomonas isabelensis | ++ |
| ECOAU1 modified | |

| | |
|---|---|
| − | No Enzymatic Activity |
| + | Enzymatic Activity |
| ++ | Strong Enzymatic Activity |

TABLE 8
Microbial Hydrogen Sulfide Assay

| Microorganism | Hydrogen Sulfide Activity |
|---|---|
| wild type | − |
| Thiomonas isabelensis | − |
| ECOAU1 mutated | |

| | |
|---|---|
| − | No H₂S Activity |
| + | H₂S Activity |
| ++ | Strong H₂S Activity |

TABLE 9
Microbial Chitinase Enzyme Assay

| Microorganism | Chitinase Enzyme Activity |
|---|---|
| wild type | + |
| Thiomonas isabelensis ECOAU1 | ++ |
| mutated | |

Legend

| | |
|---|---|
| − | No Enzymatic Activity |
| + | Enzymatic Activity |
| ++ | Strong Enzymatic Activity |

TABLE 10
Microbial Cellulase Enzyme Activity

| Microorganism | Cellulase/Hemi-Cellulase Enzyme Activity |
|---|---|
| wild type | − |
| Thiomonas isabelensis | ++ |
| ECOAU1 mutated | |

Legend

| | |
|---|---|
| − | No Enzymatic Activity |
| + | Enzymatic Activity |
| ++ | Strong Enzymatic Activity |

TABLE 11
Microbial Ligninase Enzyme Activity

| Microorganism | Ligninase Enzyme Activity |
|---|---|
| wild type | − |
| Thiomonas isabelensis | ++ |
| ECOAU1 mutated | |

Legend

| | |
|---|---|
| − | No Enzymatic Activity |
| + | Enzymatic Activity |
| ++ | Strong Enzymatic Activity |

TABLE 12
Most Abundant Sugars Found In Nature

| Microorganism | Glucose | Xylose | Sucrose | Fructose | Lactose | Ga [symbol] |
|---|---|---|---|---|---|---|
| wild type | − | − | + | − | + | |
| Thiomonas isabelensis | ++ | ++ | ++ | ++ | ++ | |
| ECOAU1 mutated | | | | | | |

| Microorganism | Arabinose | Glycerol | Malic Acid | Citric Acid |
|---|---|---|---|---|
| wild type | − | + | + | − |
| Thiomonas isabelensis | ++ | ++ | ++ | ++ |
| ECOAU1 mutated | | | | |

| Legend | |
|---|---|
| − | No Metabolic Activity |
| + | Metabolic Activity |
| ++ | Strong Metabolic Activity |

[symbol]

indicates data missing or illegible when filed

TABLE 13
Most Abundant Complex Organic Polymers Found In Nature

| | Cellulose | Lignin | | | | |
|---|---|---|---|---|---|---|
| | (Hemi- | (Ligno- | | | | |
| Microorganism | Cellulose) | Cellulose) | Starch | Pectin | Chitin | Keratin * |
| wild type | − | − | − | − | + | − |
| Thiomonas isabelensis | ++ | ++ | ++ | ++ | ++ | ++ |
| ECOAU1 mutated | | | | | | |

| Legend | |
|---|---|
| − | No Metabolic Activity |
| + | Metabolic Activity |
| ++ | Strong Metabolic Activity |

TABLE 14
Nitrate Reductase Assay (NH3 → NO3— →NO2—)

| Microorganism | Nitrate Reductase |
|---|---|
| wild type | − |
| Thiomonas isabelensis | ++ |
| ECOAU1 mutated | |

Legend

| | |
|---|---|
| − | No Metabolic Activity |
| + | Metabolic Activity |
| ++ | Strong Metabolic Activity |

TABLE 15
Diverse Nitrogen Sources in Nature

| | | Uric | | | | |
|---|---|---|---|---|---|---|
| Microorganism | Urea | Acid | Nitrate | Ammonia | Putrescine | Xanthine |
| wild type | − | − | − | + | − | − |
| Thiomonas | ++ | ++ | ++ | ++ | ++ | ++ |
| isabelensis | | | | | | |
| ECOAU1 | | | | | | |
| mutated | | | | | | |

| Legend | |
|---|---|
| − | No Metabolic |
| | Activity |
| + | Metabolic |
| | Activity |
| ++ | Strong |
| | Metabolic |
| | Activity |

TABLE 16
Diverse Phosphorus and Sulfur Sources in Nature

| | | | Inositol | | Cysteine | Methionine |
|---|---|---|---|---|---|---|
| | | Pyro- | Hexa- | | Amino | Amino |
| Microorganism | Phosphate | Phosphate | Phosphate | Sulfate | acid | acid |
| wild type | − | − | − | + | − | − |
| Thiomonas | ++ | ++ | ++ | ++ | ++ | ++ |
| isabelensis | | | | | | |
| ECOAU1 mutated | | | | | | |

| Legend | |
|---|---|
| − | No Metabolic Activity |
| + | Metabolic Activity |
| ++ | Strong Metabolic |
| | Activity |

TABLE 17
Diverse Saline and Osmotic Environments

| | | | | Ammonium | Sodium | NaCl 6% + β |
|---|---|---|---|---|---|---|
| | NaCl | KCl | Urea | Sulfate pH 8 | Nitrate pH 8 | Glutamic |
| Microorganism | 6%/wt | 6%/wt | 6%/wt | 100 mM | 100 mM | Acid |
| wild type | − | − | − | + | − | − |
| Thiomonas isabelensis | ++ | ++ | ++ | ++ | ++ | ++ |
| ECOAU1 mutated | | | | | | |

No Metabolic Activity
Metabolic Activity
Strong Metabolic Activity

TABLE 18
Diverse pH Environments

| | | pH 4.5 + | | | | pH 9.5 + |
|---|---|---|---|---|---|---|
| Microorganism | PH4 | Urea | pH 7 | Ph 9 | Ph 9.5 | Urea |
| wild type | + | − | + | − | − | − |
| Thiomonas isabelensis | ++ | ++ | ++ | ++ | ++ | ++ |
| ECOAU1 mutated | | | | | | |

No Metabolic Activity
Metabolic Activity
Strong Metabolic Activity

TABLE 19
Bioreactor control and treatment

| | CONTROL | | | T-1 | | | T-2 | | | T-3 | | |
|---|---|---|---|---|---|---|---|---|---|---|---|---|
| | KeV | Photon | Weight | KeV | Photon | Weight | KeV | Photon | Weight | KeV | Photon | Weight |
| | Range | # | % | Range | # | % | Range | # | % | Range | # | % |
| Ti | 0.4403296 | 542 | | 0.4408997 | 497 | | 0.4402218 | 522 | | 0.4406376 | 514 | |
| | 0.4603469 | 53 | | 0.460917 | 79 | | 0.4602391 | 89 | | 0.4606549 | 75 | |
| | 4.50385 | 138 | | 4.50442 | 358 | | 4.503742 | 169 | | 4.504158 | 171 | |
| | 4.924214 | 27 | | 4.924784 | 61 | | 4.924106 | 32 | | 4.904505 | 33 | |
| TOTAL | | 760 | 4.5444 | | 995 | 9.9898 | | 812 | 5.9171 | | 793 | 5.6936 |
| Zn | 1.000815 | 56 | | 1.001385 | 47 | | 1.000707 | 53 | | 1.001123 | 60 | |
| | 8.627422 | 26 | | 8.627993 | 415 | | 8.627315 | 673 | | 8.62773 | 640 | |
| TOTAL | | 82 | 0.2342 | | 462 | 2.5783 | | 726 | 5.0095 | | | 5.1252 |
| Au | 9.708359 | 8 | | 9.708929 | 28 | | 9.708251 | 35 | | 9.708667 | 37 | |
| TOTAL | | 8 | 0 | | 28 | 0 | | 35 | 0 | | 37 | 0 |

| T-4 | | | T-5 | | | Bioreactor Bottom | | | BioReactor 8/18 | | |
|---|---|---|---|---|---|---|---|---|---|---|---|
| KeV | Photon | Weight | KeV | Photon | Weight | KeV | Photon | Weight | KeV | Photon | Weight |
| Range | # | % | Range | # | % | Range | # | % | Range | # | % |
| 0.440737 | 494 | | 0.440487 | 461 | | 0.4404392 | 532 | | 0.4402841 | 534 | |
| 0.460754 | 76 | | 0.460504 | 77 | | 0.4604566 | 69 | | 0.4603014 | 84 | |
| 4.504257 | 184 | | 4.504007 | 169 | | 4.50396 | 147 | | 4.503804 | 161 | |
| 4.904604 | 48 | | 4.904354 | 31 | | 4.904306 | 32 | | 4.904151 | 26 | |
| | | 6.0828 | | | 5.4494 | | 780 | 4.2111 | | 805 | 4.5031 |
| 1.001222 | 49 | | 1.000972 | 38 | | 1.000925 | 49 | | 1.00077 | 72 | |
| 8.62783 | 748 | | 8.62758 | 914 | | 8.627532 | 2009 | | 8.627377 | 2046 | |
| | 797 | 5.5145 | | | 6.2656 | | 2058 | 15.1936 | | 2118 | 13.9725 |
| 9.708766 | 41 | | 9.708516 | 56 | | 9.708468 | 55 | | 9.708313 | 68 | |
| | 41 | 0.6348 | | 56 | 0.4467 | | 58 | 0 | | 68 | 0.3778 |

TABLE 20

| | | | ECOAU1 |
|---|---|---|---|
| | Untreated | Industrial Standard | Treated |
| | Control | Acidithiobacillus | Thomonas |
| | Rock Ore As Is | feroxidans | isabelensis |
| MgO | 3.159 | 2.7838 | 1.7399 |
| Al₂O₃ | 17.899 | 10.2713 | 1.6181 |
| Si | 83.347 | 70.2226 | 38.2929 |
| P | 0.0693 | 0.0272 | <LOD |
| S | 2.2189 | 1.9328 | 0.5288 |
| K₂O | 2.7445 | 2.1506 | 0.2892 |
| Ca | 10.993 | 5.1817 | 11.5252 |
| Ti | 2917 | 0.2181 | 0.0432 |
| V | 0.006 | 0.005 | 0.0039 |
| Cr | 0.011 | 0.0099 | 0.0033 |
| Mn | 0.0078 | 0.0054 | 0.012 |
| Fe | 1.9345 | 1.5882 | 0.6074 |
| Co | 0.02101 | 0.0029 | <LOD |
| Ni | 0 | 0 | <LOD |
| Cu | 0.001 | 0.001 | 0.0022 |
| Zn | 0.0093 | 0.0075 | 0.0028 |
| Ga | 0 | 0 | <LOD |
| As | 0.0533 | 0.0364 | 0.0111 |
| Se | 0.0003 | 0.0003 | 0.0002 |
| Rb | 0.0089 | 0.0073 | 0.0017 |
| Sr | 0.0099 | 0.0082 | 0.0117 |
| Y | 0.0069 | 0.0022 | 0.0035 |
| Zr | 0.0344 | 0.0122 | 0.0127 |
| Nb | 0 | 0.003 | <LOD |
| Ru | 0 | 0 | <LOD |
| Rh | 0 | 0 | 0 |
| Pd | 0 | 0 | <LOD |
| In | 0 | 0 | <LOD |
| Sn | 0 | 0 | 0.0059 |
| Sb | 0.045 | 0.015 | 0.0168 |
| Ba | 0.0099 | 0.0093 | 0.0079 |
| La | 0.0089 | 0.0075 | <LOD |
| Hf | 0 | 0.0002 | <LOD |
| Au | 0.159 | 0.6312 | 4.113 |
| TI | 0 | 0.008 | 0.0055 |
| Pb | 0.0009 | 0.0009 | <LOD |
| Th | 0 | 0 | <LOD |

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “isolated” in the context of an isolated bacterial strain, is one which is altered or removed from the natural state through human intervention.

What is claimed is:

1. An isolated bacterial strain Thiomonas isabelensis (ECOAU001), which has been designated Accession number NRRL No. B-67995, deposited in accordance with the Budapest Treaty at the Agricultural Research Service Culture Collection (USDA, ARS, 1815 North University Street, Peoria, IL, 61064) on Nov. 13, 2020.

2. A method of extracting, amplifying, or producing precious metals and/or rare earth metals comprising inoculating a solid substrate with the isolated bacterial strain of claim 1.

3. A method of extracting, amplifying, or producing precious metals and/or rare earth metals comprising inoculating a geological substrate with the isolated bacterial strain of claim 1.

4. A method of extracting, amplifying, or producing precious metals and/or rare earth metals comprising inoculating a liquid substrate with the isolated bacterial strain of claim 1.

5. The method of claim 2, wherein the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm.

6. The method of claim 2, wherein the solid substrate is obtained from one or more of a terrestrial, aquatic or marine source.

7. The method of claim 6, wherein, the solid substrate is one or more of soil, biofilm, sediment, native metal rock and sludge residue.

8. The method of claim 3, wherein the geological substrate is one or more of sandstone, limestone, shale, coal, chalk deposit formations, refractory rock ore.

9. The method of claim 4, wherein the liquid substrate is obtained from one or more of a waste waters, sludge waters, saltwater, freshwater, irrigation systems, ponds, lakes, rivers, and estuaries source.

10. The method of claims 2, wherein the solid substrate is disinfected and/or sterilized prior to inoculation with the bacterial strain.

11. The method of claim 2, further comprising adding a fertilizer, nutrient and/or by product composition one or more times to the substrate after inoculation with the bacterial strain.

12. The method of claim 2, comprising allowing sufficient time for the bacterial strain to colonize and exponentially grow on or in the substrate.

13. The method of claim 4, wherein an anodic and cathodic LED having a wavelength generator set at a range of 2.0-22.0 KHz is used in the liquid substrate.

14. The method of claim 2, comprising harvesting the precious metals and/or rare earth metals by separating the precious metals and/or rare earth metals from the substrate.

15. A composition comprising an isolated bacterial strain Thiomonas isabelensis (ECOAU001), about 0.01%/Wt gold emulsion, about 0.01%/Wt gold nanoparticles, about 0.01%/Wt. adenosine triphosphate, about 1%/Wt. seaweed extract, about 0.5%/Wt. humic acid, about 0.5%/Wt. mixture of nitrogen, phosphate, potassium and micronutrient mixture, about 0.1% light and mid chain hydrocarbon mixture, about 0.25%/Wt. green solvent, about 0.75%/Wt. sugar, about 1.0%/Wt. substrate selected from a gold, precious metal group, platinum metal group, rare earth metal group and combinations thereof, and a carrier selected from water or agarose.

16. The composition of claim 15, wherein the water carrier is selected from deionized water, distilled water, filtered water, well water, tap water, fresh water, sea water, brackish water, mineralized water, carbonated water, saline water, ionically charged water, ionized water, hydrogen water and combinations thereof.

17. The composition of claim 15, further comprising one or more components selected from biosolvents ethyl lactate, ATP, ADP, pyrophosphate, soy based solvents, chemical solvents, green solvents, range of organic acids, lactic acid, malic acid, ascorbic acid, alkanes, alkenes, alkynes, saturates, aromatics, resinoids, asphaltenes, light, mid chain and heavy chain hydrocarbons, sodium nitrate, sodium nitrite, ethanol, sulfur, sulfate, sulfite, nitrogen, chemical surfactants (ionic, anionic, cationic, zwitterionic surfactants), polymers (low, mid, heavy chains), biosurfactants, glycolipids, rhamnolipids (J1 and J2), glycerin, propylene glycol, carbon sugars, dextrose, galactose, sucrose, fructose, complex carbohydrates, starch, cellulose, lignin, keratin, proteins and amino acids, manures, composts, green waste, sludge material, humic and fulvic acids, coal ash and coal derived waste, alumina cytokines and seaweed extracts.

18. The method of claim 3, wherein the geological substrate is disinfected and/or sterilized prior to inoculation with the bacterial strain.

19. The method of claim 3, wherein the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm.

20. The method of claim 4, wherein the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm.
